(19) United States
(12) Patent Application Publication
(10) Pub. No.: US 2019/0156935 A1
(43) Pub. Date: May 23, 2019

(54) PATIENT MONITOR AND PHYSIOLOGICAL INFORMATION MANAGEMENT SYSTEM

(71) Applicant: NIHON KOHDEN CORPORATION, Tokyo (JP)

(72) Inventors: Mitsuhiro OURA, Tokorozawa-shi (JP); Sou KUMAGAI, Tokorozawa-shi (JP); Wataru MATUZAWA, Tokorozawa-shi (JP); Nobuyuki YASUMARU, Tokorozawa-shi (JP); Kazuya NAGASE, Tokorozawa-shi (JP); Hiroshi TORIGAI, Tokyo (JP); Naoki FUKUSHIMA, Tokyo (JP)

(21) Appl. No.: 16/193,511

(22) Filed: Nov. 16, 2018

(30) Foreign Application Priority Data
Nov. 20, 2017 (JP) 2017-222351

(51) Int. Cl. G16H 30/20; G06F 17/30; G16H 40/63; A61B 8/00; A61B 8/08; A61B 90/00

(52) U.S. Cl. CPC G16H 30/20; G06F 17/30221; G06F 17/3028; A61B 90/361; A61B 8/463; A61B 8/5261; G16H 40/63

(57) ABSTRACT

A patient monitor is connectable to an imaging unit. The patient monitor includes a folder creating section and a file operating section. When a first event occurs, the folder creating section newly creates a first folder in a first hierarchy of a file system. When a second event occurs, the folder creating section newly creates a second folder in the first folder that is last created. The file operating section stores an image file relating to image information that is supplied from the imaging unit, in the second folder last created by the folder creating section.

### CROSS-REFERENCE TO RELATED APPLICATIONS

This application claims priority to Japanese Patent Application No. 2017-222351 filed on Nov. 20, 2017, the content of which is incorporated herein by reference.

### BACKGROUND

The presently disclosed subject matter relates to a patient monitor, physiological information management system, and program which handle an image file.

An ultrasonic inspection apparatus is widely used for knowing a condition of a chest, an abdomen, or the like of a patient. Recently, also a configuration which, simultaneously with an ultrasonic inspection, measures various vital signs (blood pressure, body temperature, respiration, pulse rate, arterial oxygen saturation, and the like) of the patient has been proposed.

For example, WO2009/138902A1 discloses a system in which an ultrasonic probe head can be connected to a patient monitor (FIG. 1 of WO2009/138902A1). The system can simultaneously process both an ultrasonic image acquired by the ultrasonic probe head, and a vital parameter (vital sign) of the subject.

According to the system of WO2009/138902A1, a taken image can be easily monitored in an environment where a vital sign is to be monitored. Therefore, the system may be used in various sites (e.g. a surgery room, an intensive care unit (ICU), a general ward, and an inspection room).

Because of a wide variety of utilization sites, the patient who is to be monitored by the patient monitor may frequently be changed to another patient, and there may be no sufficient time to organize image files obtained by imaging procedures (e.g., in a case where the patient monitor is used in an emergency site). In such a case, image files may be stored in a file system of the patient monitor without being adequately classified, and, after a lapse of time from the imaging, it may be difficult to refer to a required image file (a taken image is hardly found).

### SUMMARY

According to illustrative aspects of the presently disclosed subject matter, a patient monitor which is connectable to an imaging unit (a camera or an ultrasonic probe) would adequately classify and manage image files.

According to exemplary embodiments of the presently disclosed subject matter, a patient monitor is connectable to an imaging unit. The patient monitor includes a folder creating section and a file operating section. When a first event occurs, the folder creating section newly creates a first folder in a first hierarchy of a file system. When a second event occurs, the folder creating section newly creates a second folder in the first folder that is last created. The file operating section stores an image file relating to image information that is supplied from the imaging unit, in the second folder last created by the folder creating section.

According to exemplary embodiments of the presently disclosed subject matter, new folders are created every time when the first events and second events occur, and image files are stored in the second folder which is recently created. That is, the patient monitor creates folder configurations depending on the first events and the second events, and stores the image files in a position related to the most recent second event. Since the file configuration and the storing of the image files are carried out in accordance with the events, it would be easy for users to access required image files.

According to exemplary embodiments of the presently disclosed subject matter, a patient monitor can adequately classify and manage image files.

### BRIEF DESCRIPTION OF THE DRAWINGS

FIG. 1 is a schematic view illustrating the appearance of a physiological information management system according to an embodiment of the presently disclosed subject matter;

FIG. 2 is a view illustrating a usage mode of the physiological information management system;

FIG. 3 is a block diagram illustrating a configuration of the physiological information management system;

FIG. 4 is a conceptual view illustrating a file system in the physiological information management system.

### DETAILED DESCRIPTION

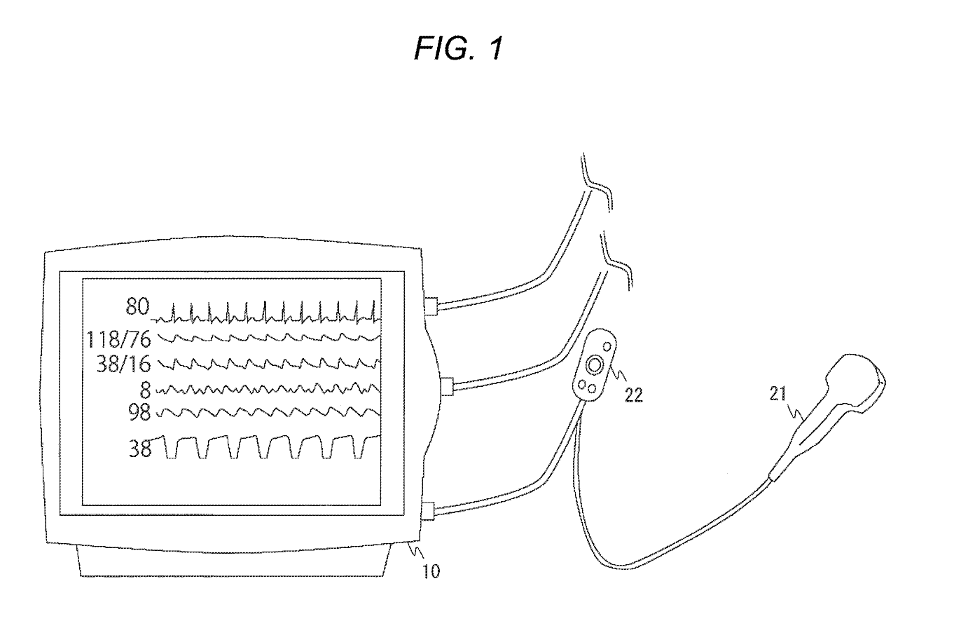

Hereinafter, an embodiment of the presently disclosed subject matter will be described with reference to drawings. FIG. 1 is a schematic view illustrating an appearance of a physiological information management system 1 of the embodiment. The physiological information management system 1 includes a patient monitor 10, and an imaging unit 20 which is detachable from the patient monitor 10. The concept of the patient monitor 10 includes a bedside monitor, a portable medical telemetry device, a defibrillator having a function of measuring, for example, an electrocardiogram, and the like. Namely, the patient monitor 10 can be interpreted as various medical devices which measure vital signs. In the following, the description will be made assuming that the patient monitor 10 is a so-called bedside monitor. In the embodiment, the imaging unit 20 has a configuration having: an ultrasonic probe head 21 which can acquire image information relating to an ultrasonic wave; and a camera-equipped remote controller 22. The imaging unit 20 may include another mode (e.g., a mode formed only by the ultrasonic probe head 21) as far as the unit includes an imaging function which can acquire some kind of image.

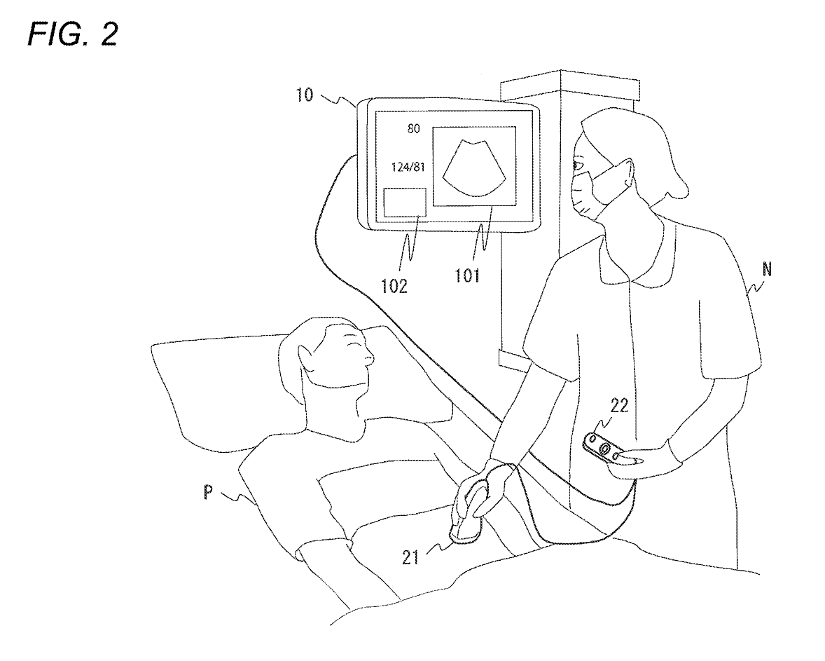

FIG. 2 is a view illustrating a usage mode of the physiological information management system 1 of the embodiment. In the embodiment, the patient monitor 10 which has a mode of the wall-mounted type will be illustrated.

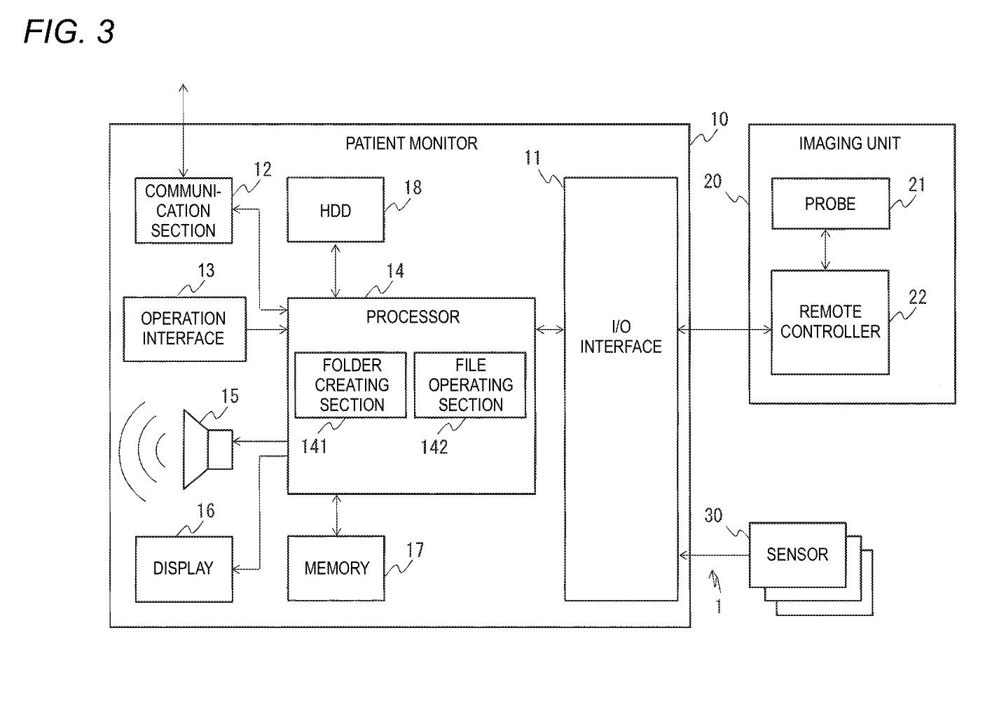

The patient monitor 10 measures various vital signs based on vital sings signals which are obtained from various sensors 30 (described later with reference to FIG. 3) connected to a patient P. The sensors 30 connected to the patient P are various sensors which are used for measuring vital signs. For example, the sensors 30 include: a cuff used for measuring the blood pressure; electrodes (disposal electrodes, clip electrodes, and the like) used for measurement of an electrocardiogram, and the like; an SpO2 probe; a mask for measuring respiration; etc. The vital signs which are the measurement targets are, for example, the blood pressure, the body temperature, the respiration rate, the arterial oxygen saturation, an electrocardiogram, and the pulse rate.

In the example of FIG. 2, the nurse N operates the ultrasonic probe head 21 by the right hand while grasping the remote controller 22 by the left hand, to acquire an ultrasonic image of the abdomen of the patient P. The patient monitor 10 displays an ultrasonic image 101 which is acquired by the ultrasonic probe head 21, and a taken image 102 which is taken by the remote controller 22, on a display. The patient monitor 10 may further display measurement waveforms of various vital signs (the blood pressure, the body temperature, the respiration, the pulse rate, the arterial oxygen saturation, and the like) of the patient P together with measurement values of the vital signs, on the display.

The imaging unit 20 is requested to include a configuration where the unit can be connected to the patient monitor 10. Namely, the connection is not limited to the illustrated wired connection, and the imaging unit 20 may transmit and receive data to and from the patient monitor 10 via wireless connection.

The patient monitor 10 includes a connection port (a so-called plug-in port) which is to be connected to the various sensors 30. The imaging unit 20 is a device which can be connected and disconnected to and from the connection port. For example, the imaging unit 20 and the patient monitor 10 may be connected to each other via a universal serial bus (USB), or via another arbitrary connector.

Then, the detailed configuration and operation of the physiological information management system 1 will be described with reference to FIG. 3. FIG. 3 is a block diagram focusing on the configuration of the physiological information management system 1 of the embodiment. As described above, the sensors 30 are vital sign sensors configured to be connected (e.g., stuck) to the living body of the subject.

As described above, the imaging unit 20 includes the ultrasonic probe head 21 and the remote controller 22. The ultrasonic probe head 21 is pressed against the abdomen or the like of the patient, and transmits and receives an ultrasonic wave. The ultrasonic probe head 21 butts against the body surface of the subject, transmits an ultrasonic beam toward the body surface, and receives a signal indicative of a reflective wave from the body surface. Then, the ultrasonic probe head 21 supplies a reflection signal (the image signal) to the patient monitor 10. The ultrasonic probe head 21 may supply digital data (the ultrasonic image data) which are produced by performing various signal processes on the reflection signal, or an image file itself to the patient monitor 10. Namely, the ultrasonic probe head 21 may transmit image information (the image signal, the ultrasonic image data, the image file) relating to an ultrasonic diagnosis, to the patient monitor 10. The ultrasonic probe head 21 incorporates various circuits, processor, and the like which are necessary for transmission and reception of the ultrasonic beam.

The remote controller 22 includes an interface (e.g., buttons) for performing various settings of the patient monitor 10 and the ultrasonic probe head 21. The remote controller 22 transmits various control signals according to an input operation, to the patient monitor 10 and the ultrasonic probe head 21. The remote controller 22 further has a camera function, and is configured so as to be able to take an image of the circumference. Namely, a lens, an imaging button, and the like are disposed on the housing of the remote controller 22, and various image processing functions are incorporated in the remote controller. The remote controller 22 transmits image information (the image signal, the taken image data, the image file) relating to the camera imaging, to the patient monitor 10.

The patient monitor 10 includes an input-output interface 11, a communication section 12, an operation interface 13, a processor 14, a speaker 15, a display section 16, a memory 17, and a hard disk drive 18. The patient monitor 10 adequately includes also hardware such as peripheral circuits which are not illustrated, an internal clock, and the like.

The input-output interface 11 is configured by the above-described connection port, its peripheral circuits, etc. The input-output interface 11 supplies signals which are received from the sensors 30 and the imaging unit 20, to the processor 14. Furthermore, the input-output interface 11 transmits signals from the patient monitor 10 to the sensors 30 or the imaging unit 20.

The communication section 12 transmits and receives data to and from another device (e.g., a central monitor of an identical hospital). For example, the communication section 12 is requested to satisfy a communication standard for a wireless LAN (Local Area Network) or the like. The communication section 12 may conduct a communication process through a wired cable.

The user (mainly, the doctor or the nurse) performs an input operation on the patient monitor 10 through the operation interface 13. The operation interface 13 is configured by buttons, knobs, rotary selector, keys, or the like which are disposed on, for example, the housing of the patient monitor 10. An input through the operation interface 13 is supplied to the processor 14.

The speaker 15 outputs various annunciation sounds such as an alarm. The speaker 15 performs annunciation in accordance with the control by the processor 14.

The display section 16 is configured by the display which is disposed on the housing of the patient monitor 10, its peripheral circuits, and the like. The display section 16 displays waveforms and measurement values of various vital signs, an ultrasonic image, and the like in accordance with the control by the processor 14. The display section 16 further displays various images (an ultrasonic image, a peripheral image) which are taken by the imaging unit 20, in accordance with the control by the processor 14.

A configuration (such as that similar to a so-called touch panel) in which the operation interface 13 and the display section 16 are integrated with each other may be employed.

The memory 17 functions as a working area in the case where the processor 14 executes a program. The hard disk drive 18 stores various programs (including system software and various kinds of application software), and data (measurement data including measurement values and measurement waveforms of vital signs, days and times when the measurement data are measured, ultrasonic images which will be described later, peripheral images, and the like). The hard disk drive 18 may be built-in to the patient monitor 10, or externally disposed.

The processor 14 controls operations (controls of the measurement via the sensors 30, reflections of various settings, captures of ultrasonic images, recordings of measurement values of vital signs, control of display on the display section 16, and the like) of the patient monitor 10. The process of the processor 14 is realized by developing a program which is read from the hard disk drive 18, in the memory 17, and then executing the program.

The programs may be stored by using a non-transitory computer readable medium of any one of various types, and then supplied to the computer. The non-transitory computer readable medium includes tangible storage media of various types. Examples of the non-transitory computer readable medium are a magnetic recording medium (e.g., a flexible disk, a magnetic tape, and a hard disk drive), a magneto-optical recording medium (e.g., a magneto-optical disk), a CD-ROM (Read Only Memory), a CD-R, a CD-R/W, a semiconductor memory (e.g., a mask ROM, a PROM (Programmable ROM), an EPROM (Erasable PROM), a flash ROM, and a RAM (random access memory)). Alternatively, the programs may be supplied to the computer by means of a transitory computer readable medium of any one of various types. Examples of the transitory computer readable medium include an electrical signal, an optical signal, and an electromagnetic wave. The transitory computer readable medium can supply the programs to the computer through a wired communication path such as an electric wire or an optical fiber, or a wireless communication path.

A part of processes of the processor 14, and peripheral processes may be realized by electronic circuits which are not illustrated. The processes of the processor 14, such as display of vital signs are performed in a similar manner as those in a usual patient monitor.

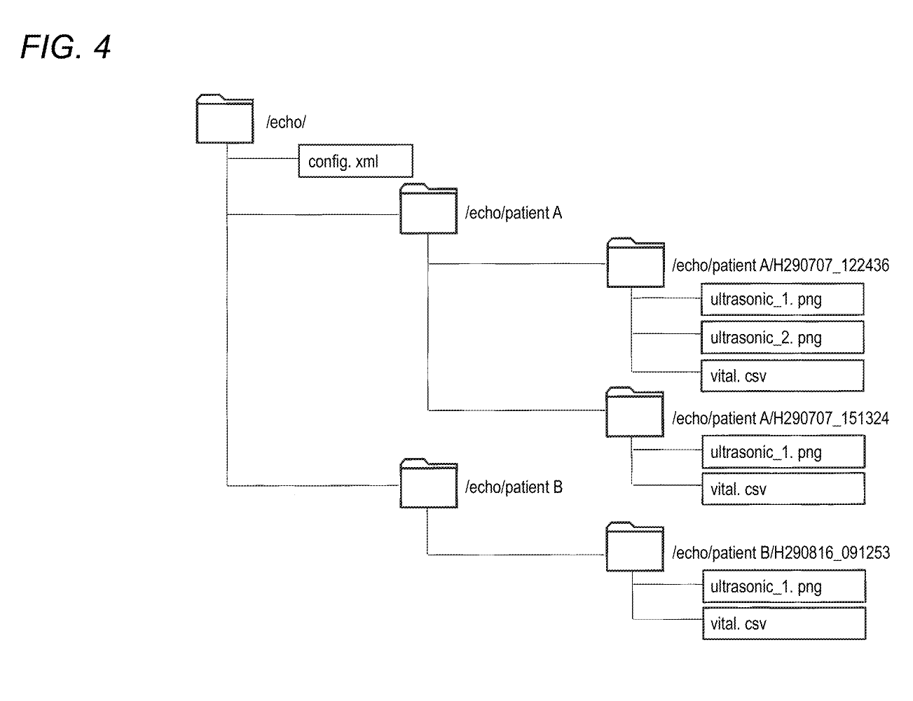

A folder creating section 141 and file operating section 142 in the processor 14 manages folder creation on a file system, and controls storing of an image file. Hereinafter, the management and the control will be described in detail adequately referring to FIG. 4.

In the case where a first event occurs, the folder creating section 141 creates a new first folder in a first hierarchy of the file system. The first event associated with a change of a patient to be monitored (measured) by the patient monitor 10. For example, the event may include at least one of a bed assignment for a new patient (e.g., a completion of an operation of a bed assignment button of the patient monitor 10 and an input of associated patient information, an update of patient information (the name of the patient, the sex, and the like), a power-on operation after an elapse of a predetermined power-off time period (e.g., one month or longer)), an operation of an inspection-start button for a new patient. In accordance with an input operation performed on the operation interface 13 or the like, the folder creating section 141 detects the first event. When such a first event occurs, the folder creating section 141 creates a new folder (the first folder) in a specific hierarchy (the first hierarchy) of the file system. In the example of FIG. 4, in the case where an event of changing the monitor target to patient A occurs, the folder creating section 141 creates a new folder in “/echo/” (the first hierarchy, “/echo/” in the example in FIG. 4). The hierarchy (the first hierarchy) in which a folder is to be created in the case where an event relating to a change of a patient occurs may be arbitrarily designated by the user.

In the creation of a folder, the folder creating section 141 preferably sets the folder name by using information identifying the patient who is designated as the new monitor target by the patient monitor 10. For example, the information identifying the patient may be the name (full name), patient ID, patient attribute (a combination of the age and the sex), date and time of bed assignment, ID of the patient monitor 10, and the like which are input in the process of bed assignment. In the example of FIG. 4, the folder creating section 141 creates a folder named “patient_A” in “/echo/”.

In the case where a second event occurs, the folder creating section 141 creates a second folder in the first folder which is last created. The second event is associated with a start of a new series of imagings (may include imaging by an ultrasonic wave and/or imaging by the camera). For example, the second event may include at least one of “an input of image information after elapse of a predetermined time period (e.g., fifteen minutes or longer) from the previous input of image information from the imaging unit 20”; “another imaging after an operation of an imaging end button”; “input of image information in a state where no folder exists in the last created first folder (in the example of FIG. 4, a state where no folder exists in folder “patient_A”); or the like. The folder creating section 141 may be enabled to detect the occurrence of the second event by, for example, appropriately referring date and time information which is managed by the operating system, and storing the time period of the previous image information input (writing of the time in the hard disk drive 18). Alternatively, the folder creating section 141 may detect the occurrence of the second event by referring a counter which is reset at every input of image information.

In the example of FIG. 4, it is assumed that the folder which, nearest to the occurrence of the second event, is created in “/echo/” is “patient_A”. When the second event occurs, the folder creating section 141 creates a new folder (second folder) in folder “patient_A”.

In the creation of the folder, preferably, the folder creating section 141 sets the folder name by using the current date and time or information which can identify the imaging sequence. In the example of FIG. 4, the folder creating section 141 sets “H290707_122436” indicating the current date and time (e.g. 12:24:36, July 7 of the 29th year of the Heisei era) as the folder name. Alternatively, the folder creating section 141 may set a name including a serial number indicating the creation sequence in folder “patient_A”, such as “patient_A_1”, or “patient_A_2”, or the like, as the folder name.

Next, the operation of the folder operating section 142 will be described. The image information (the image signal, the taken image data, the image file) is input from the imaging unit 20 in the patient monitor 10. When the image signal or the taken image data are input, the processor 14 creates an image file (e.g. a bitmap file) based on the signal and the data.

The file operating section 142 stores the image file (e.g. a bitmap file) in a folder of the file system. Specifically, the folder operating section 142 stores the image file in the second folder (the folder that is created as a result of the occurrence of the second event) last created by the folder creating section 141. In advance of the storing of the image file performed by the folder operating section 142, here, the folder creating section 141 performs the above-described creation of the second folder in the case where the input of the image information relating to the image file corresponds to the second event. When image information is input, namely, the folder creating section 141 creates the second folder in the case where the input corresponds to the second event, and thereafter the folder operating section 142 performs an operation of storing the image file. In the case where the input of the image information does not correspond to the second event, the folder operating section 142 performs an operation of storing the image file without performing further operations.

In the case where, in FIG. 4, the last created second folder is “/echo/patient_A/H290707_122436”, the folder operating section 142 stores image file “ultrasonic_1.png” in “/echo/patient_A/H290707_122436”. The folder operating section 142 may appropriately acquire information such as the imaging date and time of the image file, the imaging mode (B-mode, M-mode, or the like), and the imaging sequence, from the imaging unit 20, and set the file name by using the acquired information.

When the image file is to be stored, the folder operating section 142 may cause, together with the image file, also a vital sign file (in the example of FIG. 4, “vital.csv”) into which data of vital signs at the imaging timing of the image file are written, to be stored. The file format of the vital sign file may be arbitrary, and, in place of a CSV file, an XML file or the like may be used. In the vital sign file, for example, the blood pressure, respiration rate, body temperature, and the like of the patient at the imaging timing are written. For example, the folder operating section 142 may create the vital sign file by reading the imaging date and time from the attribute information of the image file, and reading data of vital signs corresponding to the imaging date and time from the hard disk drive 18. In place of the creation of the vital sign file, the folder operating section 142 may embed data of vital signs at the imaging time, into the image file (information such as “body temperature=36.0°” may be written overlappingly with the taken image). Alternatively, the folder operating section 142 may create only one vital sign file for each patient, and write measurement values of vital signs while being associated with the date and time information, in the vital sign file. The folder operating section 142 may set data of vital signs as the property of the file. Even in the case where the data are set as the property of the file, it is possible to know, together with the taken image, also the condition of the patient at the imaging time, and the imaging conditions. Namely, the folder operating section 142 is requested to include a mode in which measurement data of vital signs at the imaging time are stored in an arbitrary file, a database, the property of a file, or the like.

When each image file is to be stored, moreover, the folder operating section 142 may write attribute information (various kinds of information such as the type of the ultrasonic probe head 21, the gain/depth of an ultrasonic image, and the zoom state of a camera image) of the image file, and information of the folder configuration (information including the stored positions of image files), in a configuration file (“config.xml” in FIG. 4). The configuration file may be placed at an arbitrary position of the file system. When each image file is to be stored, furthermore, the folder operating section 142 may write the attribute information of the image file, and the information of the folder configuration in a database or the like. Namely, the folder operating section 142 is requested to include a configuration where, when each image file is to be stored, the attribute information of the image file, and the information of the folder configuration are stored in an arbitrary mode (such as the file creation and the writing into the database).

With reference to FIG. 4, processes relating to the creation of a folder and the storing of an image file will be described in chronological order. In the following description, it is assumed that the second event is “image information is input after elapse of a predetermined time period (e.g., fifteen minutes or longer) from the previous input of image information from the imaging unit 20.”

It is assumed that the monitor target of the patient monitor 10 is set to patient A (the first event (a change of a patient to be monitored) occurs). The folder creating section 141 detects the first event, and creates a folder named “patient_A” in “/echo/”.

It is assumed that an ultrasonic inspection of patient A is performed at 12:24:36, July 7 of the 29th year of the Heisei era (the second event (a new series of imagings) occurs). For example, the information relating to the start of the ultrasonic inspection may be determined based on a comparison between the input date and time of new image information (image file) and the previous input date and time, or on an imaging start signal which is received from the imaging unit 20. The folder creating section 141 detects the second event, and creates a folder named “H290707_122436” in “/echo/patient_A/”.

It is assumed that, during the ultrasonic inspection, two image files (“ultrasonic_1.png” and “ultrasonic_2.png”) are input. The folder operating section 142 stores “ultrasonic_1.png”, “ultrasonic_2.png”, and “vital.csv” in the last created folder (“/echo/patient_A/H290707_122436”). In the case where the time period from the input of “ultrasonic_1.png” to that of “ultrasonic_2.png” is short (e.g., within fifteen minutes), it is not deemed that the second event occurs.

Next, it is assumed that a new ultrasonic inspection of patient A is performed at 15:13:24, July 7 of the 29th year of the Heisei era. In this case, two or more hours have elapsed from the previous imaging (12:24:36, July 7 of the 29th year of the Heisei era), and hence this is treated as an occurrence of the second event. The folder creating section 141 detects the second event, and creates a folder named “H290707_151324” in “/echo/patient_A/”.

It is assumed that, during the ultrasonic inspection, one image file (“ultrasonic_1.png”) is created. The folder operating section 142 stores “ultrasonic_1.png” and “vital.csv” in the last created folder (“/echo/patient_A/H290707_151324”).

Then, it is assumed that a process of changing a patient to be monitored by the patient monitor 10 is performed. For example, it is assumed that the operation interface 13 of the patient monitor 10 is operated, and a process of bed assignment for patient B is performed. In other words, it is assumed that the monitor target of the patient monitor 10 is changed from patient A to patient B (the first event (a change of a patient to be monitored) occurs). The folder creating section 141 detects the first event, and creates a folder named “patient_B” in “/echo/”.

It is assumed that an ultrasonic inspection of patient B is performed at 9:12:53, August 16 of the 29th year of the Heisei era (the second event (a new imaging) occurs). The folder creating section 141 detects the second event, and creates a folder named “H290816_091253” in “/echo/patient_B/”.

It is assumed that, during the ultrasonic inspection, one image file (“ultrasonic_1.png”) is created. The folder operating section 142 stores “ultrasonic_1.png” and “vital.csv” in the last created folder (“/echo/patient_B/H290816_091253”).

The creation of a folder by the folder creating section 141 is not always required to be performed immediately after an event. For example, the folder creating section 141 may create a folder after it is determined that an image file is to be stored in a new folder. Specifically, the folder creating section 141 may continuously create folder “patient_A” and folder “H290707_122436” after the storing of “ultrasonic_1.png” is determined. That is, the folder creating section 141 may create a folder at an arbitrary timing as far as folders are created in the above-described sequence.

When a new folder is to be created, the folder creating section 141 may set a folder(s) in the same hierarchy to be write-protected. In the case where a folder named “H290707_151324” is to be newly created in “/echo/patient_A/”, for example, the folder creating section 141 may set other folders (such as “/echo/patient_A/H290707_122436”) to be write-protected. This prevent a file modification after a series of inspections is ended, from occurring, and correct information can be continued to be held.

Although only image files relating to an ultrasonic wave have been treated in the description with reference to FIG. 4, the kind of files is not always limited to this. Also an image file relating to the imaging by the remote controller 22 may be similarly treated. The second folder which is created in the first folder may have a plurality of hierarchies. For example, the folder creating section 141 may create a folder named “H290707_122436” in “/echo/patient_A/”, and, below the folder, create folder “ultrasonic_image” (“/echo/patient_A/H290707_122436/ultrasonic_image”) and folder “camera_image” (“/echo/patient_A/H290707_122436/camera_image”). In this case, the folder operating section 142 may store an image file in one of the folders in accordance with whether the image is an ultrasonic image or a camera image.

Then, the effect of the patient monitor 10 of the embodiment will be described with reference to FIG. 4. When a change of a patient occurs, the folder creating section 141 creates a folder (“/echo/patient_A”, “/echo/patient_B”) relating to the new patient. In the case where a new series of imagings occurs, moreover, the folder creating section 141 creates a folder for the series of imagings in the folders for respective patients (“/echo/patient_A/H290707_122436”, “/echo/patient_A/H290707_151324”, “/echo/patient_B/H290816_091253”). The folder operating section 142 stores the image file in the folder (the second folder, for example, “/echo/patient_A/H290707_122436”) last created in the folder (the first folder) for patients.

Namely, the folder creating section 141 creates a new folder each time when the first event (patient change) and the second event (start of a series of imaging) occur. The folder operating section 142 stores an image file in the last created folder for the series of imagings. Therefore, a folder configuration in which patients and various inspections are classified is automatically created, image files are stored while being classified. Therefore, the user (e.g., a medical person such as the doctor) can easily access a desired image file.

A folder (the first folder) which is created in the first hierarchy has a folder name using the identification information (e.g., “patient_A”) of the patient to be monitored. A folder (the second folder) which is created in a folder (the first folder) created in in the first hierarchy has a folder name (e.g., “H290707_122436”) using the information indicating the current time or the creation sequence. The user can easily know the time when each of files in the folder was obtained, and the person who conducted the inspection (measurement) in which the file was obtained.

Moreover, a vital sign file (which is “vital.csv” in FIG. 4, and which may have an arbitrary file format) that holds measurement values of the vital signs during imaging may be stored together with image files. When referring to the vital sign file, a medical person can know in more detail the condition of the patient at the time of imaging.

Although the invention conducted by the inventor has been specifically described based on the embodiment, the invention is not limited to the above-described embodiment, and it is a matter of course that various changes can be made without departing from the spirit of the invention.

Although, in the above description, for example, the first event is an event indicating that the patient to be monitored by the patient monitor is changed, and the second event is an event indicating that a new series of imagings is started, the kinds of the events are not limited to the above. The folder creating section is requested to detect two arbitrary kinds of events, and construct a folder configuration according to the detected event. As a result, a folder configuration and storing of image files according to events are realized, and therefore the user can easily access an image file.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A patient monitor connectable to an imaging unit, the patient monitor comprising:
a folder creating section configured to newly create a first folder in a first hierarchy of a file system in response to an occurrence of a first event, and to newly create a second folder in response to an occurrence of a second event in the first folder that is last created; and
a file operating section configured to store an image file relating to image information that is supplied from the imaging unit, in the second folder last created by the folder creating section.

2. The patient monitor according to claim 1, wherein the folder creating section is configured to set a folder name of the first folder based on information identifying a patient who is monitored by the patient monitor.

3. The patient monitor according to claim 1, wherein the folder creating section is configured to set a folder name of the second folder based on information of a current time or information identifying a creation sequence.

4. The patient monitor according to claim 1, wherein the file operating section configured to store data of vital signs at a timing of imaging the image file, when the file operating section stores the image file.

5. The patient monitor according to claim 1, wherein the first event is associated with a change of a patient to be monitored by the patient monitor.

6. The patient monitor according to claim 5, wherein the first event includes at least one of a completion of an operation of a bed assignment button and an input of associated patient information, an update of patient information, a power-on operation after an elapse of a predetermined power-off time period, and an operation of an inspection-start button for a new patient.

7. The patient monitor according to claim 1, wherein the second event is associated with a start of a new series of imagings.

8. The patient monitor according to claim 7, wherein the second event includes at least one of an input of the image information after elapse of a predetermined time period from a previous input of the image information from the imaging unit, imaging after an operation of an imaging end button, and an input of the image information in a state where no folder exists in the first folder that is last created.

9. The patient monitor according to claim 1, wherein the imaging unit includes an ultrasonic probe head configured to acquire information relating to an ultrasonic image, and a camera configured to acquire peripheral image information.

10. The patient monitor according to claim 9, wherein the image information supplied from the imaging unit relates to the ultrasonic image or an imaging by the camera.

11. The patient monitor according to claim 1, wherein, when a new folder is to be created, the folder creating section is configured to set another folders in an identical hierarchy of the new folder to be write-protected.

12. The patient monitor according to claim 1, wherein the file operating section is configured to store attribute information of the image file, and information of a folder configuration.

13. A physiological information management system comprising:
a patient monitor configured to measure vital signs of a patient; and
an imaging unit configured to produce a taken image and to send the taken image to the patient monitor,
wherein the patient monitor includes:
a folder creating section configured to newly create a first folder in a first hierarchy of a file system in response to an occurrence of a first event, and to newly create a second folder in response to an occurrence of a second event in the first folder that is last created; and
a file operating section configured to store an image file relating to image information that is supplied from the imaging unit, in the second folder last created by the folder creating section.

14. A non-transitory computer readable medium storing a program which, when executed by a computer, causes the computer to execute a process comprising:
creating a new first folder in a first hierarchy of a file system in a patient monitor which is connectable to an imaging unit when a first event occurs, and creating a second folder in the first folder that is last created when a second event occurs; and
storing an image file relating to image information that is supplied from the imaging unit, in the second folder which is last created.
